(19) United States
(12) Patent Application Publication
(10) Pub. No.: US 2026/0207329 A1
(43) Pub. Date: Jul. 23, 2026

(54) COMMISSURE ATTACHMENT MEMBER FOR A PROSTHETIC VALVE

(71) Applicant: Edwards Lifesciences Corporation, Irvine, CA (US)

(72) Inventors: Oleksandr Barannyk, Irvine, CA (US); Brendan Michael Dalbow, Oceanside, CA (US); Justin Mark Nubla, Irvine, CA (US)

(73) Assignee: Edwards Lifesciences Corporation, Irvine, CA (US)

(21) Appl. No.: 19/529,428

(22) Filed: Feb. 4, 2026

Related U.S. Application Data
(63) Continuation of application No. PCT/US2024/041045, filed on Aug. 6, 2024.
(60) Provisional application No. 63/531,527, filed on Aug. 8, 2023.

(51) Int. Cl. A61F 2/24

(52) U.S. Cl. CPC A61F 2/2418; A61F 2220/0033; A61F 2220/0058; A61F 2230/0004; A61F 2230/0054

(57) ABSTRACT

Commissure attachment members for a prosthetic valve are disclosed. A prosthetic heart valve can include a frame, a plurality of leaflets disposed on an inside of the frame, each leaflet comprising opposing side portions, and a plurality of commissure attachment members. Each commissure attachment member comprises two leaflet attachment portions that are spaced laterally apart from one another and are coupled to respective side portions of adjacent leaflets to form a commissure, where the two leaflet attachment portions are configured to move laterally away from each other when the leaflets open under a forward flow of blood through the prosthetic valve and move laterally toward each other when the leaflets close under a reverse flow of blood.

### CROSS-REFERENCE TO RELATED APPLICATIONS

This application is a continuation of PCT Application No. PCT/US2024/041045, filed Aug. 6, 2024, which claims the benefit of U.S. Provisional Patent Application No. 63/531,527, filed Aug. 8, 2023, the entire contents of each of which are incorporated by reference herein.

### FIELD

The present disclosure relates to prosthetic heart valves, including frames for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a radially expandable and compressible cylindrical metal (or wire) frame or stent and prosthetic leaflets mounted inside the frame. Commissures can be formed by connecting pairs of commissure tabs (or adjacent sides) of adjacent leaflets to each other and to struts of the frame (for example, commissure windows formed in the frame or to struts forming a cell of the frame).

During operation of the valve in vivo, the leaflets inside the frame open during systole and close during diastole. The opening for blood to flow through the open leaflets during systole can be referred to as the geometric orifice area (GOA).

### SUMMARY

Described herein are prosthetic heart valves, delivery apparatuses, and methods for implanting prosthetic heart valves. In particular, described herein are examples of movable or expandable commissure attachment members that are configured to be part of and/or secured to a frame of the prosthetic valve. Each commissure attachment member can comprise attachment portions for attaching end portions or commissure tabs of adjacent leaflets thereto, thereby forming a commissure of the prosthetic valve. The commissure attachment members can be configured to hold the end portions of adjacent leaflets adjacent one another during diastole (for example, due to an inward bias of the attachment member) and flex or move outward to a wider or more open position during systole, thereby allowing the leaflets to open wider (increasing the geometric orifice area, GOA, of the leaflets). As such, the attachment members and prosthetic valves disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, the prosthetic heart valve can comprise a frame comprising a plurality of interconnected struts defining a plurality of circumferentially extending rows of cells arranged between an outflow end and an inflow end of the frame, where the plurality of circumferentially extending rows of cells comprises a first row of first cells disposed at the outflow end.

In some examples, the valvular structure can comprise a plurality of leaflets disposed within the frame, wherein each leaflet comprises opposing commissure tabs disposed on opposite sides of the leaflet and a cusp edge portion extending between the opposing commissure tabs.

In some examples, the prosthetic heart valve can comprise one or more commissure attachment members, each commissure attachment member comprising a first end portion comprising two spaced apart leaflet attachment portions, and a second end portion secured to a portion of struts of the plurality of interconnected struts of the frame.

In some examples, for each commissure attachment member, a first commissure tab of a first of two adjacent leaflets is secured to a first leaflet attachment portion of the two leaflet attachment portions and a second commissure tab of a second of the two adjacent leaflets is secured to a second leaflet attachment portion of the two leaflet attachment portions, thereby forming a commissure of the prosthetic heart valve.

In some examples, each commissure attachment member is configured with a laterally inward bias such that the two leaflet attachment portions are held adjacent one another in a first position and move laterally outward and away from one another to a second position in response to a force over a threshold applied to the two leaflet attachment portions.

In some examples, each commissure attachment member is secured to struts of the frame such that the two leaflet attachment portions are disposed in a first cell of the first row of first cells.

In some examples, each commissure attachment member is secured to struts of the frame such that the two leaflet attachment portions are disposed outside the frame and extend axially beyond the outflow end of the frame.

In some examples, a prosthetic heart valve comprises a frame comprising a plurality of interconnected struts defining a plurality of circumferentially extending rows of cells arranged between an outflow end and an inflow end of the frame, where the plurality of circumferentially extending rows of cells comprises a first row of first cells disposed at the outflow end. The prosthetic heart valve further comprises a plurality of leaflets disposed on an inside of the frame, where each leaflet comprises opposing sides on opposite sides of the leaflet and a cusp edge portion extending between the opposing sides. The prosthetic heart valve further comprises a commissure attachment member comprising a first end portion comprising two leaflet attachment portions that are spaced laterally apart from one another and define an open end of the commissure attachment member, where each leaflet attachment portion is coupled to one opposing side of a respective leaflet of two adjacent leaflets of the plurality of leaflets, and where the first end portion is disposed within a first cell of the row of first cells. The commissure attachment member further comprises a second end portion defining a closed end of the commissure attachment member, where the second end portion is secured to a portion of struts of the plurality of interconnected struts that are disposed upstream of the outflow end of the frame. The commissure attachment member is configured to move between a closed position where the two leaflet attachment portions are held adjacent one another with a first spacing therebetween, and an open position where the two leaflet attachment portions move laterally away from one another with a larger, second spacing therebetween.

In some examples, a prosthetic heart valve comprises a frame comprising a plurality of interconnected struts defining a plurality of circumferentially extending rows of cells arranged between an outflow end and an inflow end of the frame, where the plurality of circumferentially extending rows of cells comprises a first row of first cells disposed at the outflow end. The prosthetic heart valve further comprises a plurality of commissure attachment members, each commissure attachment member comprising a first end portion comprising two spaced apart leaflet attachment portions, and a second end portion secured to a portion of struts of the plurality of interconnected struts, where the commissure attachment member is configured with a laterally inward bias such that the two leaflet attachment portions are held adjacent one another in a first position and move laterally outward and away from one another to a second position in response to a force over a threshold applied to the two leaflet attachment portions. The prosthetic heart valve further comprises a plurality of leaflets disposed within the frame, where each leaflet comprises opposing commissure tabs disposed on opposite sides of the leaflet and a cusp edge portion extending between the opposing commissure tabs, and where at each commissure attachment member, a first commissure tab of a first of two adjacent leaflets is secured to a first leaflet attachment portion of the two leaflet attachment portions and a second commissure tab of a second of the two adjacent leaflets is secured to a second leaflet attachment portion of the two leaflet attachment portions, thereby forming a commissure of the prosthetic heart valve.

In some examples, a prosthetic heart valve comprises a frame comprising a plurality of interconnected struts defining a plurality of circumferentially extending rows of cells arranged between an outflow end and an inflow end of the frame, where the plurality of circumferentially extending rows of cells comprises a first row of first cells disposed at the outflow end. The prosthetic heart valve further comprises a plurality of leaflets disposed on an inside of the frame, where each leaflet comprises opposing sides on opposite sides of the leaflet and a cusp edge portion extending between the opposing sides. The prosthetic heart valve further comprises a plurality of commissure attachment members, each commissure attachment member comprising a first end portion comprising two leaflet attachment portions that are spaced laterally apart from one another and define an open end of the commissure attachment member, where each leaflet attachment portion is coupled to one opposing side of a respective leaflet of two adjacent leaflets of the plurality of leaflets. Each commissure attachment member further comprises a second end portion defining a closed end of the commissure attachment member, where the second end portion is secured to a portion of struts of the plurality of interconnected struts that at least partially define the outflow end of the frame and the first row of first cells, where the two leaflet attachment portions extend axially away from and beyond the outflow end of the frame, and where the commissure attachment member is configured to move between a closed position where the two leaflet attachment portions are held adjacent one another with a first spacing therebetween, and an open position where the two leaflet attachment portions move laterally away from one another with a larger, second spacing therebetween.

In some examples, a prosthetic heart valve comprises a frame and a plurality of leaflets disposed on an inside of the frame. Each leaflet comprises opposing commissure tabs. The prosthetic heart valve further comprises a plurality of commissure attachment assemblies coupled to the frame. Each commissure assembly supports two commissure tabs of adjacent leaflets to form a commissure. Each commissure attachment assembly comprises a main body and a connector positioned on opposite sides of the frame. One of the main body and the connector includes a plurality of protrusions and the other of the main body and the connector includes a plurality of cavities sized to receive the protrusions.

In some examples, a prosthetic heart valve comprises one or more of the components recited in Examples 1-14, 16-23, 25-33, 42-45 and 47 below.

An assembly can comprise a prosthetic heart valve and a delivery apparatus.

In some examples, the delivery apparatus can comprise a balloon.

In some examples, the prosthetic heart valve can be any one of the prosthetic heart valves outlined above, and where the prosthetic heart valve is radially collapsible to a collapsed configuration and radially expandable to an expanded configuration, and where the collapsed prosthetic heart valve can be mounted around the balloon and radially expanded to the expanded configuration with the balloon inside a patient's body.

In some examples, the delivery apparatus can comprise one or more mechanical actuators that apply an expansion force to the prosthetic valve.

In some examples, the delivery apparatus can comprise a sheath that is configured to move from a position covering a radially collapsed prosthetic valve to a position away from the prosthetic valve such that the prosthetic valve can self-expand to its functional size.

In some examples, an assembly comprises one or more of the components recited in Examples 14 and 24 below.

A commissure attachment member can comprise a first end portion comprising two spaced apart leaflet attachment portions.

In some examples, the commissure attachment member can comprise a second end portion that is configured with a laterally inward bias such that the two leaflet attachment portions are held adjacent one another in a first position and move laterally outward and away from one another to a second position in response to a force over a threshold applied to the two leaflet attachment portions.

In some examples, the two leaflets attachment portions are configured to be coupled to respective commissure tabs of two adjacent leaflets of a prosthetic heart valve.

In some examples, the second end portion is configured to be coupled to struts of a frame of a prosthetic heart valve.

In some examples, a commissure attachment member for a frame of a prosthetic heart valve comprises a first end portion comprising two spaced apart leaflet attachment portions, where each leaflet attachment portion is configured to be coupled to a leaflet of the prosthetic heart valve; and a second end portion that is configured to be secured to a portion of struts of the frame of the prosthetic heart valve, where the commissure attachment member is configured with a laterally inward bias such that the two leaflet attachment portions are held adjacent one another in a first position and move laterally outward and away from one another to a second position in response to a force over a threshold applied to the two leaflet attachment portions.

In some examples, a commissure attachment member comprises one or more of the components recited in Examples 34-41 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of an exemplary delivery apparatus configured to deliver and implant a radially expandable prosthetic heart valve at an implantation site.

FIG. 3 is a view of an exemplary commissure attachment member for a prosthetic valve.

FIG. 4 is a side view of a portion of an exemplary frame for a prosthetic heart valve in a radially expanded configuration, the frame comprising the commissure attachment member of FIG. 3.

FIG. 5A is a side view of the complete frame of FIG. 4 in a flattened (non-annular) state, according to an example where one empty cell is disposed between two adjacent cells that comprise the commissure attachment members.

FIG. 5B is a side view of the complete frame of FIG. 4 in a flattened (non-annular) state, according to an example where two, smaller empty cells are disposed between two adjacent cells that comprise the commissure attachment members.

FIG. 6A is a schematic of a commissure attachment member secured to two leaflets and shown in a closed position.

FIG. 6B is a schematic of the commissure attachment member secured to two leaflets and shown in an open position.

FIG. 7A is a schematic top view of a prosthetic valve including three commissure attachment members, where the commissure attachment members are shown in the closed position during diastole.

FIG. 7B is a schematic top view of the prosthetic valve of FIG. 7A, where the commissure attachment members are shown in the open position during systole.

FIG. 8 a schematic top view of a prosthetic valve showing the relative positions of the commissure attachment members and leaflets during diastole and systole.

FIG. 9 is a side view of a portion of a frame of a prosthetic valve which includes a commissure attachment member secured to the frame such that its leaflet attachment portions extend above or beyond an outflow end of the frame.

FIG. 10 is a cross-sectional view of the commissure tabs of two adjacent leaflets of a prosthetic valve secured to the leaflet attachment portions of the commissure attachment member of FIG. 3.

FIG. 11 is a perspective view of a main body of an exemplary commissure attachment member for a prosthetic valve.

FIG. 12 is a perspective view of connector of the exemplary commissure attachment member that is configured to mate with an end portion of main body of FIG. 11.

FIG. 13A is a first exploded view of the main body of FIG. 11 and connector of FIG. 12 arranged around struts of a frame for a prosthetic valve.

FIG. 13B is a second exploded view of the main body of FIG. 11 and connector of FIG. 12 arranged around struts of a frame for a prosthetic valve.

FIG. 14 is a side view of the main body of FIG. 11 with the end portion arranged around struts of the frame for the prosthetic valve, without the connector coupled thereto.

FIG. 15 is a side view of a portion of a frame for a prosthetic valve with the connector of FIG. 12 mated with the end portion of the main body of FIG. 11 around struts of the frame such that the commissure attachment member is removably attached to the frame.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can include leaflets that are secured to one another at their adjacent sides (for example, commissure tabs) to form commissures which are secured to a frame of the prosthetic heart valve. In some examples, the commissures can extend through commissure windows in the frame of the prosthetic heart valve and then be secured to the frame struts forming the commissures windows. In some examples, the commissures can be attached directly or indirectly to struts of the frame and the commissures may be static and unable to move freely during operation of the prosthetic heart valve. For example, during operation of the valve in vivo, the leaflets inside the frame open during systole and close during diastole. The opening for blood to flow through the open leaflets during systole can be referred to as the geometric orifice area (GOA). However, since the leaflet commissures are fixed to the frame (without freedom to expand and contract at the commissures), the GOA can be limited.

Described herein are various examples of a movable or expandable commissure attachment member that is configured to be part of and/or secured to a frame of a prosthetic valve. Each commissure attachment member can comprise attachment portions for attaching end portions or sides (for example, commissure tabs) of adjacent leaflets thereto, thereby forming a commissure of the prosthetic valve. The commissure attachment member can be configured with an inward bias (for example, spring-like) that holds the adjacent sides of adjacent leaflets of the commissure together (adjacent one another) during diastole. Then, during systole, in response to an increased load (force) on the leaflets, the attachment portions of the commissure attachment member can pull away from one another (open), thereby allowing the adjacent sides of the adjacent leaflets of the commissure to move away from one another, which in turn results in an enlargement in the GOA of the prosthetic valve. This can increase blood flow through the prosthetic valve and/or decrease a load (stress) on the leaflets of the prosthetic valve. This can allow leaflets of thinner material to be used.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

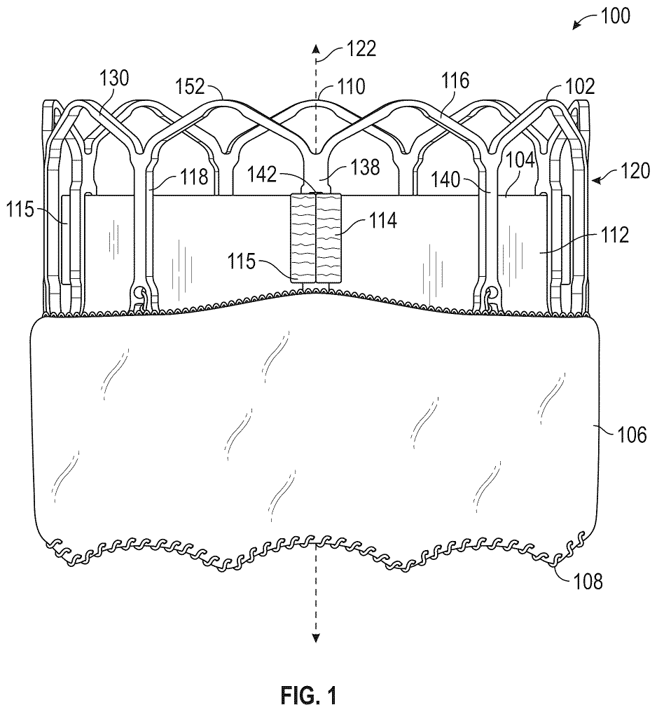

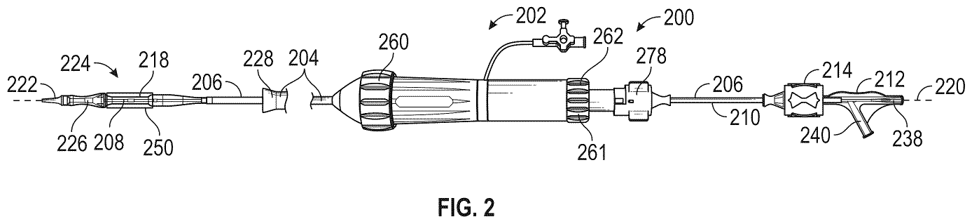

FIG. 1 illustrates an exemplary prosthetic device (for example, prosthetic heart valve) comprising a frame, leaflets secured on an inside of the frame, and an outer skirt disposed around an outer surface of the frame. In some examples, the frame can comprise commissure windows configured to receive adjacent sides of adjacent leaflets, thereby forming a commissures that are secured to the frame. The prosthetic device can be advanced through a patient's vasculature, such as to a native heart valve, by a delivery apparatus, such as the exemplary delivery apparatus shown in FIG. 2.

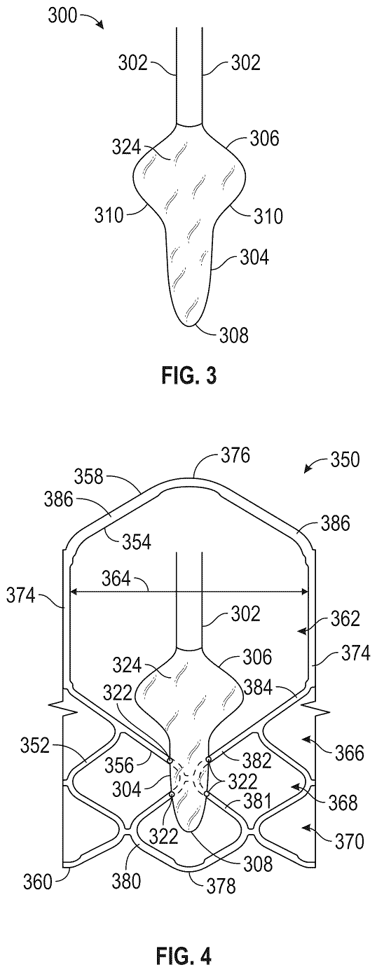

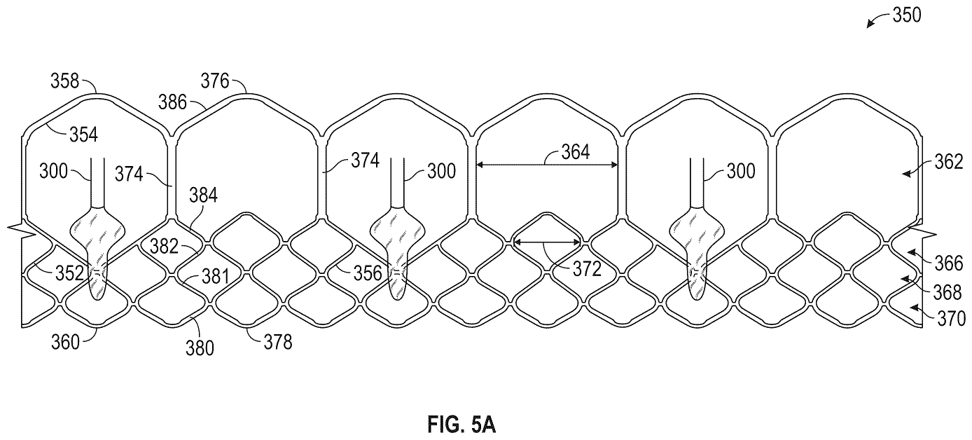

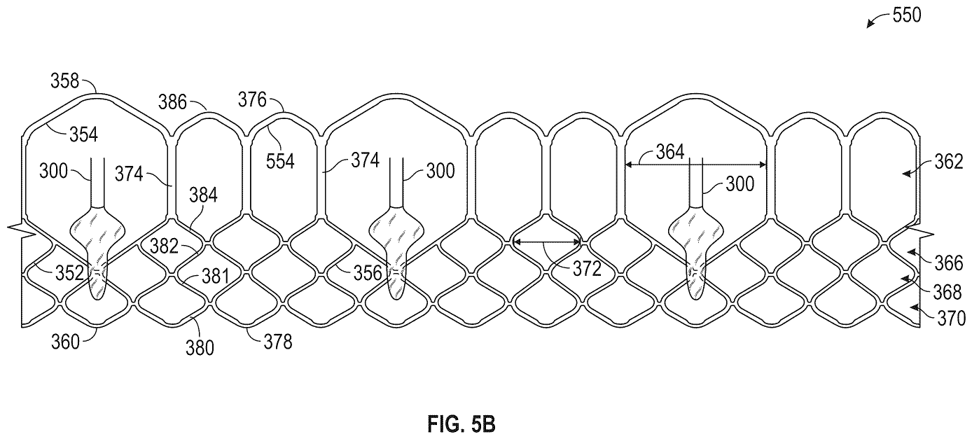

In some examples, the frame can include movable or expandable, commissure attachment members, as shown in FIGS. 4 and 5. As shown in FIG. 3, each commissure attachment member can comprise two, spaced apart, leaflet attachment portions that are configured to be secured to adjacent sides (for example, commissure tabs) of adjacent leaflets of the prosthetic device (as shown in the cross-sectional view of FIG. 10). The attachment member can further comprise a closed end disposed opposite the leaflet attachment portions. In some examples, the attachment member can be secured to the frame closer to its closed end.

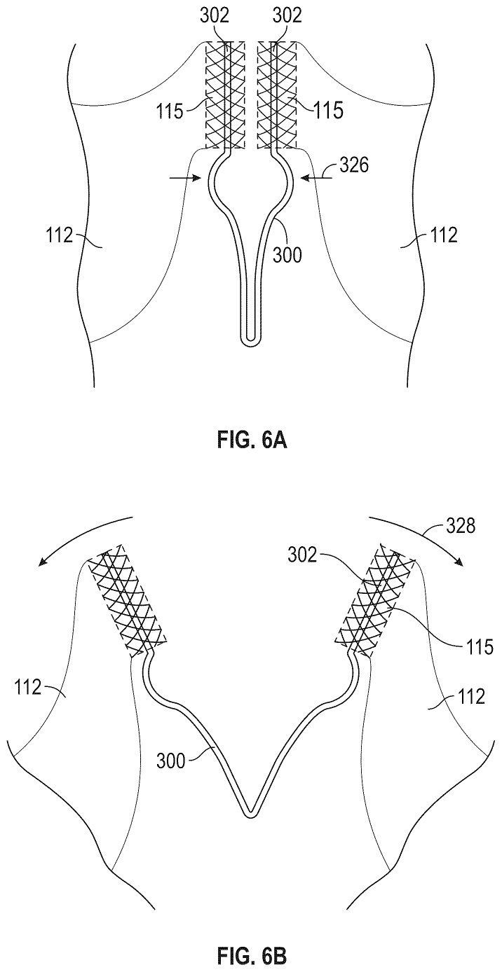

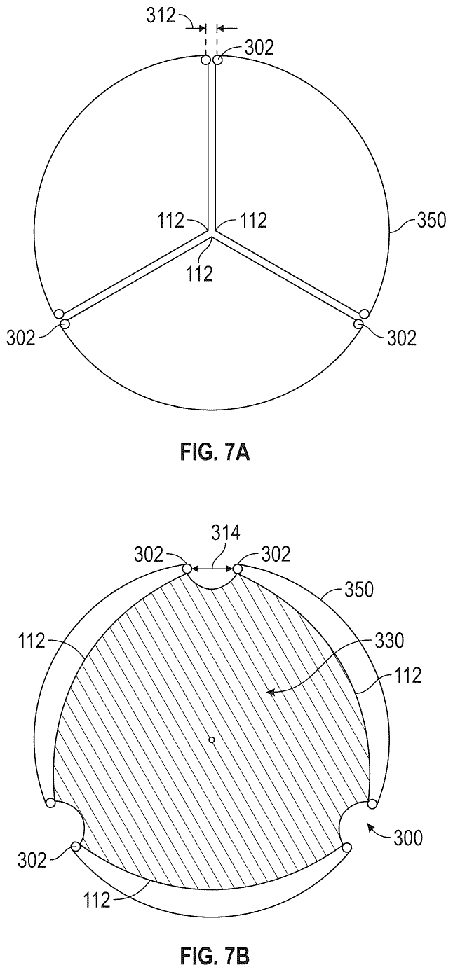

The commissure attachment member can be inwardly biased (spring-like) to a closed position shown in FIGS. 3-6A. The attachment member can be further configured to be pulled into an open position where the leaflet attachment portions move away from one another due to a force applied to the leaflets, as shown in FIG. 6B. For example, the commissure attachment members of the frame can be in the closed position during diastole (as shown in FIGS. 6A and 7A) and be pulled into the open position due to forces on the leaflets during systole (as shown in FIGS. 6B and 7B).

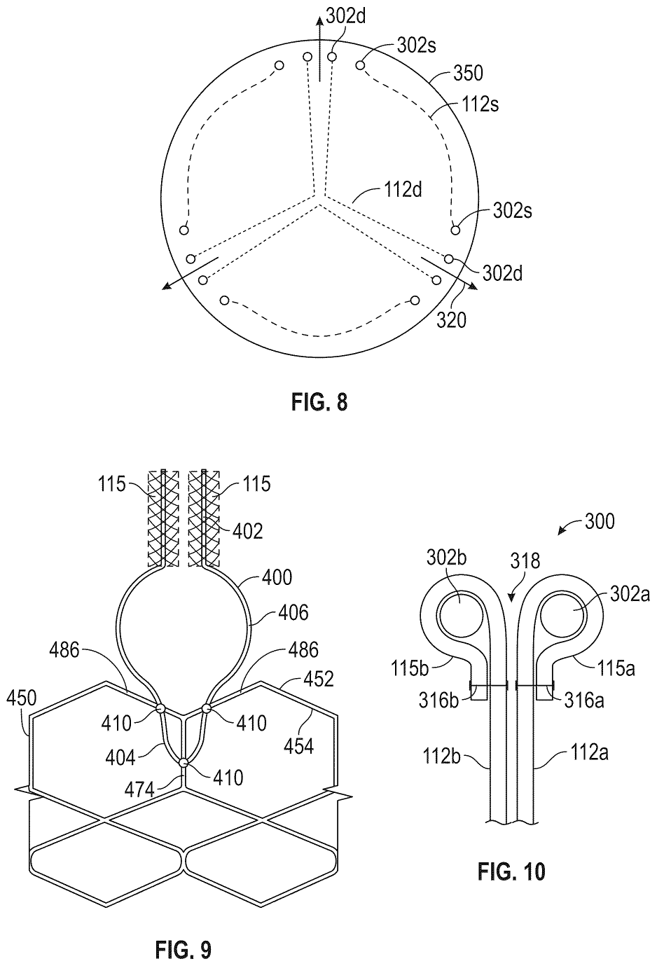

FIG. 8 shows a schematic top view of the prosthetic device and the relative positions of the commissure attachment members and leaflets during diastole and systole.

FIG. 9 shows an exemplary frame for a prosthetic device where commissure attachment members are secured to the frame such that their leaflet attachment portions extend above or beyond an outflow end of the frame.

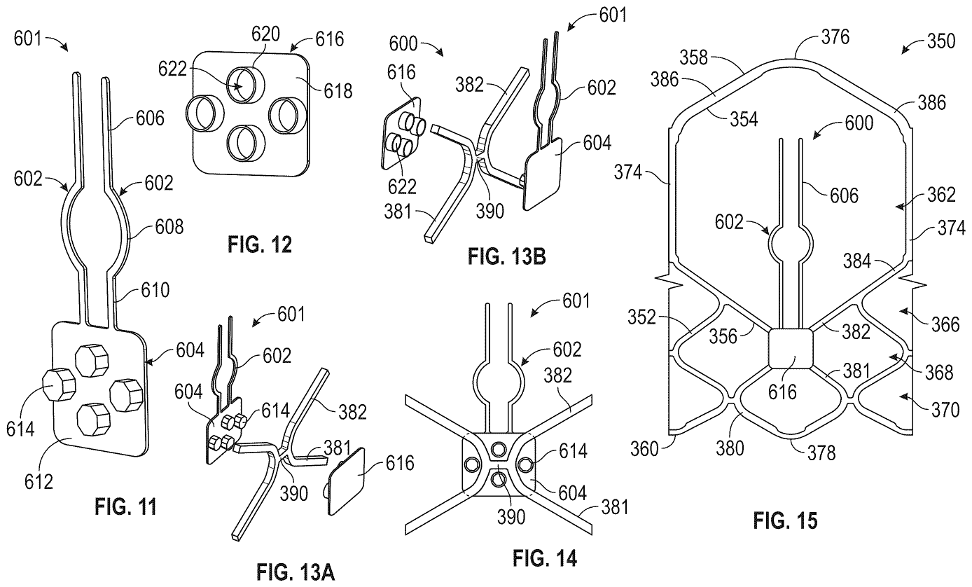

FIGS. 11-15 show an exemplary movable or expandable commissure attachment member that can be removably coupled to the frame via an interlocking interface.

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure, which can be arranged to collapse in a tricuspid arrangement (or bicuspid arrangement in some examples). The leaflets 112 can be secured to one another at their adjacent sides (for example, commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in U.S. provisional patent application No. 63/366,599 filed Jun. 17, 2022, which is incorporated by reference herein.

The frame 102 can be radially compressible and expandable between a radially compressed (or collapsed) configuration and a radially expanded configuration (the expanded configuration is shown in FIG. 1).

The frame 102 can be made of any of various suitable plastically-expandable materials (for example, stainless steel, etc.) or self-expanding materials (for example, Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIG. 1, the frame 102 can comprise a plurality of interconnected struts 116 which form multiple rows of open cells 118 between the outflow end 110 and the inflow end 108 of the frame 102. In some examples, the frame 102 can comprise three rows of cells 118 with a first (upper in the orientation shown in FIG. 1) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length than cells 118 in the remaining rows of cells.

In some examples, as shown in FIG. 1, each row of cells comprises nine cells 118. Thus, in such examples, the frame 102 can be referred to as a nine-cell frame.

In alternate examples, the frame 102 can comprise more than three rows of cells (for example, four or five) and/or more or less than nine cells per row. In some examples, the cells 118 in the first row of cells 120 may not be elongated compared to cells 118 in the remaining rows of cells of the frame 102.

The interconnected struts 116 can include a plurality of angled struts 130 arranged in a plurality of rows of circumferentially extending rows of angled struts, with the rows being arrayed along the length of the frame 102 between the outflow end 110 and the inflow end 108. The interconnected struts 116 can also include a plurality of axially extending window struts 138 (or window strut portions) and a plurality of axial (or axially extending) struts 140. The axially extending window struts 138 (which can also be referred to as axial struts that include a commissure window) define commissure windows (for example, open windows) 142 that are spaced apart from one another around the frame 102, in a circumferential direction, and which are adapted to receive a pair of commissure tabs of a pair of adjacent leaflets 112 arranged into a commissure (for example, commissure 114). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIG. 1) axial struts 140 can be positioned between, in the circumferential direction, two commissure windows 142 formed by the window struts 138. Since the frame 102 can include fewer cells per row (for example, nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 forms an axial side of two adjacent cells of the first row of cells 120.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

The frame 102 can further comprise a plurality of apex regions 152 formed at the inflow end 108 and the outflow end 110, each apex region 152 extending and forming a junction between two angled struts 130 at the inflow end 108 or outflow end 110. As such, the apex regions 152 are spaced apart from one another, in a circumferential direction at the inflow end 108 and the outflow end 110. Additional details and examples of frames for prosthetic heart valves that include apex regions can be found in PCT Application No. PCT/US2022/025687, which is incorporated by reference herein.

FIG. 2 shows a delivery apparatus 200, according to an example, that can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 2 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 2, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 2) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Application No. PCT/US2021/047056, which is incorporated by reference herein.

As introduced above and shown in FIG. 1, prosthetic valves can include leaflets (for example, leaflets 112) that are secured to one another at their adjacent sides (for example, commissure tabs) to form commissures (for example, commissures 114 in FIG. 1) which are secured to a frame of the prosthetic valve. In some examples, as shown in FIG. 1, the commissures can extend through commissure windows in the frame of the prosthetic heart valve and then be secured to the frame struts forming the commissures windows. The commissures may be static and unable to move freely during operation of the prosthetic valve (for example, when implanted in a native heart valve). For example, during operation of the valve in vivo, the leaflets inside the frame open during systole and close during diastole. The opening for blood to flow through the open leaflets during systole can be referred to as the geometric orifice area (GOA). However, since the leaflet commissure are fixed to the frame (without freedom to expand and contract at the commissures), the GOA can be limited.

Described herein are various examples of commissure attachment members for frames of prosthetic valves that are configured to expand (or move) into a more open configuration in response to pressure on the leaflets of the prosthetic valve during systole (for example, when implanted in a native heart valve). The commissure attachment members can be part of and/or secured to a frame of a prosthetic valve (such as any of the frames described herein, or a self-expanding or mechanically expandable frame). Each commissure attachment member can comprise attachment portions for attaching end portions or sides (for example, commissure tabs) of adjacent leaflets thereto, thereby forming a commissure of the prosthetic valve. The commissure attachment member can be configured with an inward bias (like a spring) that holds the adjacent sides of the adjacent leaflets of the commissure toward or adjacent one another during diastole. During systole, in response to an increased load (force) on the leaflets, the attachment portions of the commissure attachment member are pulled or forced away from one another (into an open position), thereby allowing the adjacent sides of the adjacent leaflets of the commissure to move away from one another. This can result in an enlargement in the GOA of the prosthetic valve, which can increase blood flow through the prosthetic valve and/or decrease a load (stress) on the leaflets of the prosthetic valve.

FIG. 3 depicts an exemplary commissure attachment member 300 (or commissure support member or commissure support) that is configured to support and couple to adjacent sides (for example, commissure tabs) of adjacent leaflets of a prosthetic valve (as depicted schematically in FIGS. 6A and 6B). The commissure attachment member 300 can be part of (for example, integrally formed with) or secured to (for example, laser welded) to a frame of a prosthetic valve.

An exemplary frame 350 for a prosthetic valve, which includes one or more commissure attachment members 300 attached thereto is shown in FIGS. 4 and 5A. In some examples, the frame 350 can be used in lieu of the frame 102 in the prosthetic heart valve 100 of FIG. 1. The frame 350 can be radially compressible and expandable between a radially compressed (or collapsed) configuration and a radially expanded configuration (FIGS. 4 and 5A). FIG. 4 shows a portion of the frame 350 (with one commissure attachment member 300) and FIG. 5A shows the frame 350 in a flattened (non-annular) state with three commissure attachment members 300 spaced apart across the frame (in a circumferential direction). It should be noted that the frame 350 has an annular configuration when manufactured, such as the frame shown in FIG. 1.

The frame 350 can comprise any of the plastically-expandable materials (for example, stainless steel, etc.) or self-expanding materials (for example, nickel titanium alloy (NiTi), such as nitinol) materials discussed above with reference to the frame 102.

The frame 350 can comprise a plurality of interconnected struts 352 which form multiple rows of open cells 354, 356 between an outflow end 358 and an inflow end 360 of the frame 350. In some examples, as shown in FIGS. 4 and 5A, the frame 350 can comprise four rows of cells 354, 356 with a first (upper in the orientation shown in FIGS. 4 and 5A) row of cells 362 disposed at the outflow end 358. The first row of cells 362 comprises cells 354 that are elongated in an axial direction (relative to a central longitudinal axis of the frame 350 and a direction extending between the outflow end 358 and the inflow end 360) and wider in a circumferential direction, as compared to cells 356 in the remaining rows of cells. For example, the cells 354 of the first row of cells 362 can have a longer axial length and a larger width 364 than cells 356 in the remaining rows of cells.

The increased width 364 of the cells 354 of the first row can provide a larger opening for the commissure attachment members 300. For example, the commissure attachment members 300 can extend into a center of a portion of the cells 354. The commissure attachment members 300 can replace the axially extending window struts 138 in the frame 102 of FIG. 1.

The remaining rows of cells can include a second row of cells 366, a third row of cells 368, and a fourth row of cells 370. The fourth row of cells 370 is disposed at the inflow end 360 and the second row of cells 366 is disposed between the first row of cells 362 and the third row of cells 368 (which is disposed adjacent to the fourth row of cells 370).

In some examples, as shown in FIG. 5A, each of the second row of cells 366, the third row of cells 368, and the fourth row of cells 370 comprise 12 cells 356 and the first row of cells 362 comprises six cells 354. This arrangement of differing numbers of cells between the first row of cells 362 and the remaining rows of cells is due to the width 364 of the cells 354 of the first row of cells 362 being twice as wide as a width 372 of the cells 356 of the remaining rows of cells (for example, the adjacent second row of cells 366). Thus, in some examples, each cell 354 of the first row of cells 362 can span the width of two cells 356 (such as the third row of cells 368, as depicted in FIGS. 4 and 5). Said another way, the width 364 of each cell 354 can be double the width 372 of a cell 356.

In alternate examples, the frame 350 can comprise a different number of rows of cells, such as three (for example, similar to the frame 102 of FIG. 1) or five. Additionally or alternatively, in some instances the frame 350 can include more or less than 12 and six cells per row (for example, ten and five cells per row).

The interconnected struts 352 can include a plurality of angled struts 380, 381, 382, 384, and 386 arranged in a plurality of rows of circumferentially extending rows of angled struts, with the rows being arrayed along the length of the frame 350 between the inflow end 360 and the outflow end 358. For example, the frame 350 can comprise a first row of angled struts 380 arranged end-to-end and extending circumferentially at the inflow end 360 of the frame; a second row of circumferentially extending, angled struts 381; a third row of circumferentially extending, angled struts 382; a fourth row of circumferentially extending, angled struts 384, and a fifth row of circumferentially extending, angled struts 386 at the outflow end 358 of the frame 350. The circumferentially extending rows of angled struts (as well as additional components, such as the rows of cells) can be referred to herein as being “upstream” or “downstream” of other circumferentially extending rows of angled struts. As used herein, “upstream” or “downstream” are relative to the outflow end 358 of the frame (which is the downstream end of the frame) and the inflow end 360 of the frame (which is the upstream end of the frame) and a direction of blood flow through the frame 350 (from the inflow end 360 to the outflow end 358). For example, the fourth row of angled struts 384 is disposed upstream of the fifth row of angled struts 386.

The fifth row of angled struts 386 can be connected to the fourth row of angled struts 384 by a plurality of axial (or axially extending) struts 374 (which can be configured similar to the axial struts 140 of frame 102, as described above).

Each axial strut 374 extends from a location defined by the convergence of the lower ends (ends arranged inward of and farthest away from the outflow end 358) of two angled struts 386 (which can also be referred to as an upper strut junction or upper elongated strut junction) to another location defined by the convergence of the upper ends (ends arranged closer to the outflow end 358) of two angled struts 384 (which can also be referred to as a lower strut junction or lower elongate strut junction). Each axial strut 374 forms an axial side of two adjacent cells 354 of the first row of cells 362.

In some examples, the frame 350 can further comprise a plurality of apex regions 376 formed at the outflow end 358 and plurality of apex regions 378 formed at the inflow end 360. Each apex region 378 can extend and form a junction between two angled struts 380 at the inflow end 360 of the frame 350. Each apex region 376 can extend and form a junction between two angled struts 386 at the outflow end 358 of the frame 350. The apex regions 376 and 378 can be configured the same as or similarly to the apex regions 152, as described above.

In some examples, the fourth row of angled struts 384 may not include the angled struts 384 extending into the cells 354 of the first row of cells 362 that do not have the commissure attachment members 300 therein. In such examples, the fourth row of angled struts 384 may only include the angled struts 384 connected to the axial struts 374.

FIG. 5B shows an exemplary frame 550 that includes the commissure attachment members 300, as described above. The frame 550 is the same as frame 350, except the first row of cells 362 comprises the wider cells 354 with the commissure attachment members 300 disposed therein, and narrower cells 554 disposed between the wider cells 354. For example, as shown in FIG. 5B two narrower cells 554 are disposed between each pair of adjacent wider cells 354. In some examples, this arrangement of the frame 550 may provide increased structural stability and allow the cells containing the commissure attachment member 300 to be more visible under fluoroscopy. All the other components of the frame 550 can be the same as in frame 350 and have been labeled accordingly.

Returning to FIG. 3, the commissure attachment member 300 can comprise a first end portion which comprises two spaced apart leaflet attachment portions 302, a second end portion 304 disposed opposite the first end portion, and a wider, intermediate portion 306 disposed between the leaflet attachment portions 302 and the second end portion 304.

The leaflet attachment portions 302 are each configured to be secured to one of two adjacent sides of two adjacent leaflets (for example, commissure tabs 115 of leaflets 112, as shown in FIGS. 6A and 6B). In some examples, the leaflet attachment portions 302 can be vertical (or relatively vertical or axially extending) posts, prongs, or struts around which the sides of the adjacent leaflets can be secured.

For example, in some instances, as shown in the cross-sectional view of FIG. 10, a first commissure tab 115a of a first leaflet 112a can extend through an opening 318 between first and second leaflet attachment portions 302a, 302b of the commissure attachment member 300, wrap around a first leaflet attachment portion 302a, and be secured to itself by a first fastener 316a (such as a suture). A second commissure tab 115b of a second leaflet 112b can extend through the opening 318 between the first and second leaflet attachment portions 302a, 302b of the commissure attachment member 300, wrap around a second leaflet attachment portion 302b, and be secured to itself by a second fastener 316b (such as a suture).

In some examples, the commissure tabs 115a, 115b of the leaflets 112a, 112b can wrapped and/or folded around, or otherwise attached to, the respective leaflet attachment members 102a, 102b in different ways than that shown in FIG. 10.

The intermediate portion 306 can be wider and extend laterally or circumferentially (when attached to or part of the frame 350) beyond the leaflet attachment portions 302 and the second end portion 304. For example, in some instances, the intermediate portion 306 can comprise two opposing curved portions that curve circumferentially outward from the leaflet attachment portions 302 and second end portion 304, in opposite directions from one another.

The second end portion 304 can comprise a closed end 308 (which can be referred to as an inflow end). In some examples, the commissure attachment member 300 can be referred to as having two arms 310 that are spaced (laterally) apart from one another along an entirety of the commissure attachment member 300 until they connect together at the closed end 308.

In some examples, the commissure attachment member 300 can be referred to as a V-shaped spring or attachment member comprising the closed end 308 and an open end defined by the spaced apart leaflet attachment portions 302.

In some examples, the commissure attachment member 300 can be secured to struts of the frame 350 along its second end portion 304. For example, as shown in FIG. 4, the second end portion 304 of the commissure attachment member 300 is attached to one or more of angled struts 382 and 381.

In some examples, both arms 310 in the region of the second end portion 304 of the commissure attachment member 300 can be attached to the angled struts 382 and/or 381.

In some examples, the second end portion 304 of the commissure attachment member 300 can be welded (for example, laser welded) at one or more of the weld points 322 shown in FIG. 4.

In some examples, the second end portion 304 of the commissure attachment member 300 can be welded (for example, laser welded) at each of the weld points 322 shown in FIG. 4.

In some examples, the second end portion 304 of the commissure attachment member 300 can be welded at various numbers of weld points 322, such as any combination of the weld points 322 shown in FIG. 4, or additional or different weld points than those shown in FIG. 4.

In some examples, the second end portion 304 of the commissure attachment member 300 can be integrally formed with the angled struts 382 and 381, such as extending from and between the angled struts 382 and 381, as shown in FIG. 4.

In some examples, the second end portion 304 of the commissure attachment member 300 can be secured to selected struts (for example, struts 381, 382) with sutures. Other techniques and mechanisms can be used to secure the commissure attachment member 300 to the frame, such as, rivets, pins, screws, clips, or various other types of fasteners.

The intermediate portion 306 of the commissure attachment member 300 can extend away from the angled struts 382 and into a cell 354 (as shown in FIGS. 4 and 5). The intermediate portion 306 can be unattached to the struts of the frame 350 (not directly attached to the struts of the frame 350).

The leaflet attachment portions 302 can extend further into the cell 354 toward but spaced away from the outflow end of the cell 354, as defined by the angled struts 386. In this way, the leaflet attachment portions 302 can be optimally positioned within the wider cell 354 to receive the sides (for example, commissure tabs) of two adjacent leaflets.

In some examples, as shown in FIGS. 5A and 5B, the frame 350 can comprise three commissure attachment members 300 which are spaced circumferentially apart from one another across the frame 350 (in the lateral or circumferential direction). For example, as shown in FIG. 5A, a cell 354 without a commissure attachment member 300 can be spaced between two cells 354 containing a commissure attachment member 300 therein. In some examples, as shown in FIG. 5B, two cells 554 without commissure attachment members 300 can be spaced between two cells 354 containing a commissure attachment member 300 therein. In this way, the frame 350 shown in FIG. 5A and the frame 550 shown in FIG. 5B can be adapted to support three leaflets therein.

In some examples, the commissure attachment member 300 can comprise a covering element 324 extending across the second end portion 304 and the intermediate portion 306 (as shown in FIGS. 3 and 4). The covering element 324 can be a fabric (or cloth) or polymeric material that is configured to reduce or prevent blood from passing through the commissure attachment member 300 when implanted in vivo (for example, prevent or reduce perivalvular leakage). The covering element 324 can comprise any of the fabric or polymeric materials described herein.

In some examples, the covering element 324 can be bunched or comprise excess material when the commissure attachment member 300 is in the closed position such that it can freely expand to the open position (without being restricted by the material of the covering element 324) shown in FIGS. 6B and 7B, as described further below.

In some examples, the covering element 324 may extend across less of the commissure attachment member 300 than shown in FIG. 3, such as only over the second end portion 304.

The commissure attachment member 300 is shaped and sized such that it acts as a spring element with a laterally or circumferentially inward bias (as indicated by arrows 326 in FIG. 6A). For example, the arms 310 of the commissure attachment member 300 are inwardly biased toward one another such that they are held in a resting and closed position, as shown in FIGS. 3-6A and 7A, when a force below a threshold is being applied to the leaflet attachment portions 302 (for example, during diastole).

The commissure attachment member 300 can be further configured to be pulled into an open position where the leaflet attachment portions 302 move away from one another (in the direction indicated by arrows 328 in FIG. 6B) due to a force, at or above the threshold level, being applied thereto by the leaflets (for example, due to increased blood flow through the prosthetic valve which moves a body and outflow edges of the leaflets radially outward toward the frame 350), as shown in FIG. 6B and FIG. 7B (for example, during systole).

FIGS. 7A and 7B illustrate a top view of a prosthetic valve comprising the frame 350 and three leaflets 112 attached to three commissure attachment members 300 of the frame 350. The top view of FIG. 7A shows the two leaflet attachment portions 302 of each of the commissure attachment members 300 in the closed position where the two leaflet attachment portions 302 are disposed adjacent to one another (for example, at a first lateral or circumferential spacing 312, which is a non-zero amount to accommodate the thickness of the commissure tabs). The top view of FIG. 7B shows the two leaflet attachment portions 302 of each of the commissure attachment members 300 in the open position where the two leaflet attachment portions 302 are spaced circumferentially away from one another (for example, at a second lateral or circumferential spacing 314 that is larger than the first circumferential spacing 312). FIG. 7B also depicts the enlarged GOA 330 of the prosthetic valve comprising the commissure attachment members 300 (relative to a prosthetic valve with static commissure support members, such as a commissure window).

FIG. 8 shows a schematic top view of the prosthetic valve comprising the frame 350 (or a similar frame) and the relative positions of the commissure attachment members 300 and leaflets 112 (for example, an outflow edge portion of the leaflets 112) during diastole and systole. The positions of the leaflet attachment portions 302 and the leaflets 112 during diastole are labeled as “302d” and “112d”, respectively, and the positions of the leaflet attachment portions 302 and the leaflets 112 during systole are labeled as “302s” and “112s”, respectively. A direction of blood flow through the open leaflet attachment portions 302s of the commissure attachment members 300 is shown by arrows 320 in FIG. 8. In some examples, this allows for direction a portion of blood flow into the neosinus (an artificial chamber created by native valve leaflets and prosthetic valve leaflets), which can improve neosinus washout.

During systole, the increase in fluid (blood) through the prosthetic valve causes the leaflet attachment portions 302 of the commissure attachment members 300 to open (to the points labeled 302s in FIG. 8), thereby allowing the outflow edge portions of the leaflets 112 to move to the positions labeled as 112s in FIG. 8. The opening of the commissure attachment members 300 leads to a larger GOA, thereby reducing pressure gradients.

In some examples, prosthetic valves including frames including the commissure attachment members 300, such as frame 350 (or the commissure attachment members 400 in the frame 450, as described below), can have larger leaflets (relative to a frame with static commissure support members or elements). For example, larger leaflets can have a longer outflow edge portion or “operating length”. If these larger leaflets are attached to the commissure attachment members 300 (or the commissure attachment members 400), the opening of the commissure attachment members 300 during systole would result in an even larger GOA, and thus greater flow through the prosthetic valve (in contrast to a frame with static commissure support members which would result in wrinkling of the extra material of the larger leaflets).

Any of the commissure attachment members described herein (such as commissure attachment members 300 or 400) can comprise any of the frame materials described herein. In some examples, the commissure attachment members can be made of the same material as the frame. In some examples, the commissure attachment members can comprise a different material than frame, but a material that can be fixed to a material of the frame, such as by laser welding (such as a metal wire). In some examples, the commissure attachment members can comprise a shape-memory material, such as Nitinol.

Thus, in some instances, the spring-effect described above can be achieved by the material, shape and/or a thickness (for example, a thickness of the wire) of the commissure attachment member 300. In some examples, by adjusting the material properties of the material of the commissure attachment member 300, the second circumferential spacing 314 between the leaflet attachment portions 302 can be adjusted (to a specified spacing).

Another advantage of utilizing commissure attachment members 300 (or commissure attachment members 400) (especially if they comprise a shape-memory material, such as Nitinol), is the potential to use thinner tissue (porcine or skived) for the leaflets since the commissure attachment members 300 would mitigate forces applied to the leaflet tissue. Thus, the commissure attachment members 300 can reduce stresses on the leaflets during operation of the prosthetic valve, thereby increasing a robustness and longevity of the prosthetic valve.

FIG. 9 shows a portion of an exemplary frame 450 for a prosthetic device where a commissure attachment member 400, which can be similar to the commissure attachment member 300, is secured to the frame 450 such that its leaflet attachment portions 402 extend above or beyond an outflow end 452 of the frame 450.

Similar to the commissure attachment member 300, the commissure attachment member 400 can comprise the leaflet attachment portions 402 (shown schematically as secured to commissure tabs 115 of leaflets 112 in FIG. 9), forming a first end portion, a second end portion 404, and an intermediate portion 406.

In some examples, as shown in FIG. 9, the second end portion 404 can be attached (such as by laser welding, sutures, or various other types of fasteners) to struts of the frame 450 at weld points 410. In some instances, the struts of the frame 450 to which the second end portion 404 is attached can include angled struts 486 forming the outflow end 452 of the frame 450 and two adjacent cells 454 of a first row of cells of the frame 450.

In some examples, the struts of the frame 450 to which the second end portion 404 is attached can include two angled struts 486 that at least partially define the outflow end 452 of the frame 450 and two adjacent cells 454 of a first row of first cells disposed at the outflow end 452 of the frame 450 and an axially extending strut 474 that is connected to the two angled struts 486 and defines an axial side of each of the two adjacent first cells 454.

As a result, the intermediate portion 406 and leaflet attachment portions 402 extend above or past the outflow end 452 of the frame 450.

The arrangement shown in FIG. 9 provides for a lower profile frame (for example, with a shorter axial length) with only the commissure attachment members 400 disposed above or beyond the outflow end 452 of the frame 450. This may allow for a larger V-shaped opening of the prosthetic valve that is not restricted by the circumference of the frame 450.

FIGS. 11-15 depict an exemplary commissure attachment member 600 (or commissure support member or commissure support or commissure attachment assembly) that is configured to support and couple to adjacent sides (for example, commissure tabs) of adjacent leaflets of a prosthetic valve (similar to as depicted schematically in FIGS. 6A and 6B for the commissure attachment member 300).

The commissure attachment member 600 can be similar to the commissure attachment member 300, except it is attached to the frame by an interlocking interface of the commissure attachment member 600 (instead of welding to the frame). As a result, a prosthetic valve including the commissure attachment members 600 can be easier to assemble and stress concentrations on the components (the commissure attachment members 600 and/or frame) can be reduced.

A commissure attachment member 600 attached (for example, removably attached) to the exemplary frame 350 for a prosthetic valve via the interlocking interface of the commissure attachment member 600 is shown in FIG. 11. However, in some examples, the commissure attachment member 600 can be attached to a different frame, such as the frame 550 shown in FIG. 5B or the frame 450 shown in FIG. 9.

The commissure attachment member 600 comprises a main body 601 which comprises two spaced apart arms 602 (or prongs) and a coupling portion 604, where each arm 602 extends vertically from the coupling portion 604. Each arm comprises a leaflet attachment portion 606, a wider, intermediate portion 608, and connecting portion 610 that directly extends from the coupling portion 604. The leaflet attachment portions 606 form a first end portion of the main body 601 and the coupling portion 604 forms a second end portion of the main body 601.

The leaflet attachment portions 606 are each configured to be secured to one of two adjacent sides of two adjacent leaflets (for example, commissure tabs 115 of leaflets 112, similar to as shown in FIGS. 6A and 6B for the commissure attachment member 600). In some examples, the leaflet attachment portions 606 can be vertical (or relatively vertical or axially extending) posts, prongs, or struts around which the sides of the adjacent leaflets can be secured.

The intermediate portion 308 of each arm 602 can be wider and extend laterally or circumferentially (when attached to or part of the frame 350, for example) beyond the respective leaflet attachment portion 606. For example, the intermediate portions 608 can comprise two opposing curved portions that curve circumferentially outward from the leaflet attachment portions 606, in opposite directions from one another.

The connecting portions 608 can be straight and axially aligned with the leaflet attachment portions 606, in some examples.

The coupling portion 604 can comprise a plate 612 and a plurality of spaced apart protrusions 614 extending outward from a radially facing surface of the plate 612. Each protrusion can have a shape configured to mate with a cavity in a connector 616 (or connecting plate) of the commissure attachment member 600 (which is shown alone in FIG. 12). In this way, the coupling portion 604 and the connector 616, as explained further below, can form the interlocking interface of the commissure attachment member 600.

In some examples, the protrusions 614 have a cross-sectional shape of an octagon.

In some examples, the protrusions 614 can have a cross-sectional shape of another polygon, such as hexagonal, square, triangular, or the like. In some examples, the protrusions 614 can have a circular cross-sectional profile.

As shown in FIG. 12, the connector 616 can comprises a plate 618 and a plurality of spaced apart protrusions 620, each having a hollowed out or internal cavity 622. A width (or diameter) and depth (in the radial direction) of each cavity 622 can be sized to receive a respective protrusion 614 of the coupling portion 604.

In some examples, instead of protrusions, the connector 616 can be a thicker plate with similarly shaped cavities depressed into the plate. In some examples, the connector 616 can include the protrusions 614 and the coupling portion can include the cavities 622.

In some examples, as shown in FIG. 12, the protrusions 620 can be cylindrical and the cavities 622 can each have a circular cross-section.

In some examples, each of the protrusions 620 forms a friction fit with a respective cavity 622; that is, the outer surface of each protrusion tightly engages an inner surface of a respective cavity. In some examples, the combination of the hexagonal (or other polygonal) protrusions 614 and circular cavities 622 can provide a robust interlocking interface between the coupling portion 604 and the connector 616, which can allow the commissure attachment member 600 to quickly and easily be attached and unattached to struts 381, 382 of the frame 350, as depicted in FIGS. 13A-15.

FIGS. 13A and 13B show an exploded view of the commissure attachment member 600 around a portion of the frame 350. The main body 601 and the connector 616 are placed on opposite sides of the frame. For example, the main body 601 can be positioned inside the frame and the connector 616 can be positioned outside the frame; alternatively, the main body 601 can be positioned outside of the frame and the connector 616 can be positioned inside the frame. FIG. 14 shows the main body 601 arranged around the struts 381, 382 of the frame 350, prior to attaching the connector 616 thereto. As shown in FIG. 14, the protrusions 614 (and similarly the protrusions 620 of the connector 616) are spaced apart such that struts 381, 382 and a strut junction 390 therebetween can fit in spaces between the protrusions 614 (against the plate 612).

The cavities 622 of the connector 616 are mated with the protrusions 614 of the coupling portion 604, thereby securing the connector 616 to the coupling portion 604 with the struts 381, 382, and strut junction 390 sandwiched therebetween. In some examples, when the connector 616 and the coupling portion 604 of the main body are pressed together, the connector 616 can contact the outer surface (or inner surface) of the struts and the coupling portion 604 can contact the inner surface (or outer surface) of the struts, with the protrusions 614, 620 extending through cells of the frame. As a result, the commissure attachment member 600 is securely (and removably) attached to the frame 350. In some examples, in lieu of or in addition to a friction fit between the protrusions and the cavities, an adhesive can be added between the interfacing surfaces of the protrusions and the cavities to fix the connector 616 to the coupling portion 604.

As shown in FIG. 15, when attached to the frame struts, as described above, the arms 602 extend into a cell 354 of the frame 350. The leaflet attachment portions 606 can extend further into the cell 354 toward but spaced away from the outflow end of the cell 354, as defined by the angled struts 386. In this way, the leaflet attachment portions 606 can be optimally positioned within the wider cell 354 to receive the sides (for example, commissure tabs) of two adjacent leaflets.

The commissure attachment member 600 can function similarly as the commissure attachment member 300. For example, the commissure attachment member 600 is shaped and sized such that it acts as a spring element with a laterally or circumferentially inward bias. For example, the arms 602 of the commissure attachment member 600 are inwardly biased toward one another such that they are held in a resting and closed position, as shown in FIGS. 11 and 15, when a force below a threshold is being applied to the leaflet attachment portions 606 (for example, during diastole).

The commissure attachment member 600 can be further configured to be pulled into an open position where the leaflet attachment portions 606 move away from one another due to a force, at or above the threshold level, being applied thereto by the leaflets (for example, due to increased blood flow through the prosthetic valve which moves a body and outflow edges of the leaflets radially outward toward the frame 350, for example, during systole).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve, comprising: a frame; a plurality of leaflets disposed on an inside of the frame, wherein each leaflet comprises opposing side portions; and a plurality of commissure attachment members, each comprising: two leaflet attachment portions that are spaced laterally apart from one another and are coupled to respective side portions of adjacent leaflets to form a commissure, wherein the two leaflet attachment portions are configured to move laterally away from each other when the leaflets open under a forward flow of blood through the prosthetic heart valve and move laterally toward each other when the leaflets close under a reverse flow of blood.

Example 2. The prosthetic heart valve of any example herein, particularly example 1, wherein each commissure attachment member is configured with a laterally inward bias that holds the two leaflet attachment portions toward one another when the leaflets are closed.

Example 3. The prosthetic heart valve of any example herein, particularly either example 1 or example 2, wherein each commissure attachment member is configured to move from a closed position where the two leaflet attachment portions are disposed adjacent each other to an open position where the two leaflet attachment portions are moved laterally away from each other in response to a force at or above a threshold level being applied thereto by the adjacent leaflets.

Example 4. The prosthetic heart valve of any example herein, particularly example 3, wherein each commissure attachment member is in the closed position during diastole and the open position during systole when the prosthetic heart valve is implanted in a native heart valve.

Example 5. The prosthetic heart valve of any example herein, particularly any one of examples 1-4, wherein the opposing side portions of each leaflet are opposing commissure tabs, and wherein the two leaflet attachment portions are vertical posts around which a commissure tab of each of the adjacent leaflets is secured.

Example 6. The prosthetic heart valve of any example herein, particularly any one of examples 1-5, wherein the frame comprises a plurality of interconnected struts, wherein each commissure attachment member comprises an end portion disposed opposite the two leaflet attachment portions and defining a closed end of the commissure attachment member, wherein the end portion is secured to a portion of struts of the plurality of interconnected struts that are disposed upstream of an outflow end of the frame.

Example 7. The prosthetic heart valve of any example herein, particularly example 6, wherein each commissure attachment member is V-shaped with two arms extending from the closed end, each arm comprising one of the two leaflet attachment portions.

Example 8. The prosthetic heart valve of any example herein, particularly either example 6 or example 7, wherein each commissure attachment member comprises an intermediate portion disposed between the two leaflet attachment portion and the end portion, and wherein the intermediate portion is wider and extends laterally beyond the two leaflet attachment portions and the end portion.

Example 9. The prosthetic heart valve of any example herein, particularly example 8, wherein the intermediate portion and the two leaflet attachment portions are unattached to the plurality of interconnected struts.

Example 10. The prosthetic heart valve of any example herein, particularly any one of examples 6-9, wherein the end portion of each commissure attachment member is laser welded to the portion of struts.

Example 11. The prosthetic heart valve of any example herein, particularly any one of examples 6-10, wherein the portion of struts to which the end portion is secured includes one or more angled struts that at least partially define a first cell of a first row of first cells disposed at the outflow end of the frame, and wherein the two leaflet attachment portions are disposed within the first cell.

Example 12. The prosthetic heart valve of any example herein, particularly example 6, wherein the end portion comprises a plate and a plurality of spaced apart protrusions extending outward from a radially facing surface of the plate.

Example 13. The prosthetic heart valve of any example herein, particularly example 12, wherein each commissure attachment member further comprises a connector comprising a plurality of spaced apart cavities, wherein each cavity of the plurality of spaced apart cavities is shaped to receive a respective protrusion of the plurality of spaced apart protrusions such that the connector mates with the end portion.

Example 14. The prosthetic heart valve of any example herein, particularly example 13, wherein the end portion and connector are removably coupled to one another around the portion of the struts, with the portion of struts sandwiched therebetween, thereby removably coupling the commissure attachment member to the frame.

Example 15. An assembly comprising: a delivery apparatus; and the prosthetic heart valve of any example herein, particularly any one of examples 1-11, wherein the prosthetic heart valve is radially collapsible to a collapsed configuration for delivery into a patient's body on the delivery apparatus and radially expandable to an expanded configuration inside the patient's body.

Example 16. A prosthetic heart valve, comprising: a frame comprising: a plurality of interconnected struts defining a plurality of circumferentially extending rows of cells arranged between an outflow end and an inflow end of the frame, wherein the plurality of circumferentially extending rows of cells comprises a first row of first cells disposed at the outflow end; and a plurality of commissure attachment members, each commissure attachment member comprising a first end portion comprising two spaced apart leaflet attachment portions, and a second end portion secured to a portion of struts of the plurality of interconnected struts, wherein the commissure attachment member is configured with a laterally inward bias such that the two leaflet attachment portions are held adjacent one another in a first position and move laterally outward and away from one another to a second position in response to a force over a threshold applied to the two leaflet attachment portions; and a plurality of leaflets disposed within the frame, wherein each leaflet comprises opposing commissure tabs disposed on opposite sides of the leaflet and a cusp edge portion extending between the opposing commissure tabs, and wherein at each commissure attachment member, a first commissure tab of a first of two adjacent leaflets is secured to a first leaflet attachment portion of the two leaflet attachment portions and a second commissure tab of a second of the two adjacent leaflets is secured to a second leaflet attachment portion of the two leaflet attachment portions, thereby forming a commissure of the prosthetic heart valve.

Example 17. The prosthetic heart valve of any example herein, particularly example 16, wherein in the first position the two leaflet attachment portions of each commissure attachment member are spaced laterally apart by a first amount, and wherein in the second position the two leaflet attachment portions are spaced laterally apart by a second amount that is greater than the first amount.

Example 18. The prosthetic heart valve of any example herein, particularly either example 16 or example 17, wherein the two spaced apart leaflet attachment portions are axially extending posts, relative to a central longitudinal axis of the frame, and wherein, for each commissure attachment member, the first commissure tab is secured around the first leaflet attachment portion and the second commissure tab is secured around the second leaflet attachment portion.

Example 19. The prosthetic heart valve of any example herein, particularly any one of examples 16-18, wherein the second end portion defines a closed end of the commissure attachment member, and wherein each commissure attachment has two arms extending from the closed end, each arm comprising one of the two leaflet attachment portions.

Example 20 The prosthetic heart valve of any example herein, particularly any one of examples 16-19, wherein each commissure attachment member comprises an intermediate portion disposed between the first end portion and the second end portion and that is unattached to the plurality of interconnected struts, and wherein the intermediate portion comprises two opposing curved portions that curve laterally outward from the two leaflet attachment portions, in opposite directions from one another.

Example 21. The prosthetic heart valve of any example herein, particularly any one of examples 16-20, wherein the portion of struts to which the second end portion is secured includes one or more angled struts that at least partially define the outflow end of the frame and that at least partially define the first row of first cells, and wherein the two leaflet attachment portions of each commissure attachment member extend axially away from and beyond the outflow end of the frame.

Example 22. The prosthetic heart valve of any example herein, particularly any one of examples 16-20, wherein the portion of struts to which the second end portion is secured includes one or more angled struts that at least partially define the first cell, and wherein the two leaflet attachment portions of each commissure attachment member are disposed within a respective first cell of the row of first cells.

Example 23. The prosthetic heart valve of any example herein, particularly any one of examples 16-22, wherein the second end portion is removably attached to the portion of struts by a connector that mates with the second end portion around the portion of struts.

Example 24. An assembly comprising: a delivery apparatus comprising a balloon; and the prosthetic heart valve of any example herein, particularly any one of examples 16-23, wherein the prosthetic heart valve is radially collapsible to a collapsed configuration and radially expandable to an expanded configuration, and wherein the collapsed prosthetic heart valve can be mounted around the balloon and radially expanded to the expanded configuration with the balloon inside a patient's body.

Example 25. A prosthetic heart valve, comprising a frame comprising a plurality of interconnected struts defining a plurality of circumferentially extending rows of cells arranged between an outflow end and an inflow end of the frame, wherein the plurality of circumferentially extending rows of cells comprises a first row of first cells disposed at the outflow end; a plurality of leaflets disposed on an inside of the frame, wherein each leaflet comprises opposing sides on opposite sides of the leaflet and a cusp edge portion extending between the opposing sides; and a plurality of commissure attachment members, each commissure attachment member comprising: a first end portion comprising two leaflet attachment portions that are spaced laterally apart from one another and define an open end of the commissure attachment member, wherein each leaflet attachment portion is coupled to one opposing side of a respective leaflet of two adjacent leaflets of the plurality of leaflets; and a second end portion defining a closed end of the commissure attachment member, wherein the second end portion is secured to a portion of struts of the plurality of interconnected struts that at least partially define the outflow end of the frame and the first row of first cells, wherein the two leaflet attachment portions extend axially away from and beyond the outflow end of the frame, and wherein the commissure attachment member is configured to move between a closed position where the two leaflet attachment portions are held adjacent one another with a first spacing therebetween, and an open position where the two leaflet attachment portions move laterally away from one another with a larger, second spacing therebetween.

Example 26. The prosthetic heart valve of any example herein, particularly example 25, wherein each commissure attachment member is configured with a laterally inward bias that holds the two leaflet attachment portions in the closed position.

Example 27. The prosthetic heart valve of any example herein, particularly either example 25 or example 26, wherein each commissure attachment member is configured to move from the closed position to the open position in response to a force at or above a threshold level being applied thereto by the two adjacent leaflets during systole when the prosthetic heart valve is implanted in a native heart valve.

Example 28. The prosthetic heart valve of any example herein, particularly any one of examples 25-27, wherein each commissure attachment member is V-shaped with two arms extending from the closed end, each arm comprising one of the two leaflet attachment portions.

Example 29. The prosthetic heart valve of any example herein, particularly any one of examples 25-28, wherein each commissure attachment member comprises an intermediate portion disposed between the first end portion and the second end portion, wherein the intermediate portion is wider and extends laterally beyond the two leaflet attachment portions and the second end portion.

Example 30. The prosthetic heart valve of any example herein, particularly example 29, wherein the intermediate portion is unattached to the plurality of interconnected struts and disposed axially beyond the outflow end of the frame.

Example 31. The prosthetic heart valve of any example herein, particularly any one of examples 25-30, wherein the opposing sides of each leaflet are opposing commissure tabs, and wherein the two leaflet attachment portions of each commissure attachment member are vertical posts around which a commissure tab of each of the two adjacent leaflets is secured.

Example 32. The prosthetic heart valve of any example herein, particularly any one of examples 25-31, wherein the second end portion of each commissure attachment member is laser welded to the portion of struts.

Example 33. The prosthetic heart valve of any example herein, particularly any one of examples 25-32, wherein the portion of struts to which the second end portion is secured included two angled struts of the plurality of interconnected struts that at least partially define the outflow end of the frame and two adjacent first cells of the first row of first cells and an axially extending strut of the plurality of interconnected struts that is connected to the two angled struts and defines an axial side of each of the two adjacent first cells.

Example 34. A commissure attachment member for a frame of a prosthetic heart valve, comprising: a first end portion comprising two spaced apart leaflet attachment portions, wherein each leaflet attachment portion is configured to be coupled to a leaflet of the prosthetic heart valve; and a second end portion that is configured to be secured to a portion of struts of the frame of the prosthetic heart valve, wherein the commissure attachment member is configured with a laterally inward bias such that the two leaflet attachment portions are held adjacent one another in a first position and move laterally outward and away from one another to a second position in response to a force over a threshold applied to the two leaflet attachment portions.

Example 35. The commissure attachment member of any example herein, particularly example 34, wherein in the first position the two leaflet attachment portions are spaced laterally apart by a first amount, and wherein in the second position the two leaflet attachment portions are spaced laterally apart by a second amount that is greater than the first amount.

Example 36. The commissure attachment member of any example herein, particularly example 34 or example 35, wherein the two spaced apart leaflet attachment portions are axially extending posts, and wherein each axially extending post is configured to receive a commissure tab of a respective leaflet of a pair of adjacent leaflets of the prosthetic heart valve therearound.

Example 37. The commissure attachment member of any example herein, particularly any one of examples 34-36, wherein the second end portion defines a close end of the commissure attachment member, and wherein the commissure attachment member is V-shaped with two arms extending from the closed end, each arm comprising one of the two leaflet attachment portions.

Example 38. The commissure attachment member of any example herein, particularly any one of examples 34-37, wherein each commissure attachment member comprises an intermediate portion disposed between the first end portion and the second end portion, and wherein the intermediate portion comprises two opposing curved portions that curve laterally outward from the two leaflet attachment portions and second end portion, in opposite directions from one another.

Example 39. The commissure attachment member of any example herein, particularly any one of examples 34-38, wherein the second end portion comprises a plurality of spaced apart protrusions, and wherein each commissure attachment member further comprises a connector comprising a plurality of spaced apart cavities, wherein each cavity of the plurality of spaced apart cavities is shaped to receive a respective protrusion of the plurality of spaced apart protrusions such that the connector mates with the second end portion.

Example 40. The commissure attachment member of any example herein, particularly example 39, wherein the second end portion and connector are configured to be removably coupled to one another around the portion of the struts, with the portion of struts sandwiched therebetween, thereby removably coupling the commissure attachment member to the portion of struts.

Example 41. The commissure attachment member of any example herein, particularly example 39 or 40, wherein each protrusion has a polygonal cross-section and each cavity has a circular cross-section.

Example 42. A prosthetic heart valve comprising: a frame; a plurality of leaflets disposed on an inside of the frame, wherein each leaflet comprises opposing commissure tabs; and a plurality of commissure attachment assemblies coupled to the frame, wherein each commissure assembly supports two commissure tabs of adjacent leaflets to form a commissure, wherein each commissure attachment assembly comprises a main body and a connector positioned on opposite sides of the frame; wherein one of the main body and the connector includes a plurality of protrusions and the other of the main body and the connector includes a plurality of cavities sized to receive the protrusions.

Example 43. The prosthetic heart valve of any example herein, particularly example 42, wherein the protrusions frictionally engage the cavities.

Example 44. The prosthetic heart valve of any example herein, particularly either examples 42 or 43, wherein the main body comprises two leaflet attachment portions, each connected to one of the commissure tabs.

Example 45. The prosthetic heart valve of any example herein, particularly example 44, wherein the two leaflet attachment portions are spaced laterally apart from one another and are configured to move laterally away from each other when the leaflets open under a forward flow of blood through the prosthetic heart valve and move laterally toward each other when the leaflets close under a reverse flow of blood.

Example 46. A method comprising sterilizing the prosthetic heart valve, apparatus, and/or assembly of any example.

Example 47. A prosthetic heart valve of any one of examples 1-41, wherein the prosthetic heart valve is sterilized.

The features described herein with regard to any example can be combined with other features described in any one or more of the other examples, unless otherwise stated. For example, any one or more of the features of one prosthetic valve can be combined with any one or more features of another prosthetic valve. As another example, any one or more features of one commissure attachment member be combined with any one or more features of another commissure attachment member.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame;
a plurality of leaflets disposed on an inside of the frame, wherein each leaflet comprises opposing side portions; and
a plurality of commissure attachment members, each comprising:
two leaflet attachment portions that are spaced laterally apart from one another and are coupled to respective side portions of adjacent leaflets to form a commissure, wherein the two leaflet attachment portions are configured to move laterally away from each other when the leaflets open under a forward flow of blood through the prosthetic heart valve and move laterally toward each other when the leaflets close under a reverse flow of blood.

2. The prosthetic heart valve of claim 1, wherein each commissure attachment member is configured with a laterally inward bias that holds the two leaflet attachment portions toward one another when the leaflets are closed.

3. The prosthetic heart valve of claim 1, wherein each commissure attachment member is configured to move from a closed position where the two leaflet attachment portions are disposed adjacent each other to an open position where the two leaflet attachment portions are moved laterally away from each other in response to a force at or above a threshold level being applied thereto by the adjacent leaflets.

4. The prosthetic heart valve of claim 1, wherein the opposing side portions of each leaflet are opposing commissure tabs, and wherein the two leaflet attachment portions are vertical posts around which a commissure tab of each of the adjacent leaflets is secured.

5. The prosthetic heart valve of claim 1, wherein the frame comprises a plurality of interconnected struts, wherein each commissure attachment member comprises an end portion disposed opposite the two leaflet attachment portions and defining a closed end of the commissure attachment member, wherein the end portion is secured to a portion of struts of the plurality of interconnected struts that are disposed upstream of an outflow end of the frame.

6. The prosthetic heart valve of claim 5, wherein each commissure attachment member is V-shaped with two arms extending from the closed end, each arm comprising one of the two leaflet attachment portions.

7. The prosthetic heart valve of claim 5, wherein each commissure attachment member comprises an intermediate portion disposed between the two leaflet attachment portion and the end portion, and wherein the intermediate portion is wider and extends laterally beyond the two leaflet attachment portions and the end portion.

8. The prosthetic heart valve of claim 5, wherein the end portion of each commissure attachment member is laser welded to the portion of struts.

9. The prosthetic heart valve of claim 5, wherein the portion of struts to which the end portion is secured includes one or more angled struts that at least partially define a first cell of a first row of first cells disposed at the outflow end of the frame, and wherein the two leaflet attachment portions are disposed within the first cell.

10. The prosthetic heart valve of claim 5, wherein the end portion comprises a plate and a plurality of spaced apart protrusions extending outward from a radially facing surface of the plate, and wherein each commissure attachment member further comprises a connector comprising a plurality of spaced apart cavities, wherein each cavity of the plurality of spaced apart cavities is shaped to receive a respective protrusion of the plurality of spaced apart protrusions such that the connector mates with the end portion.

11. The prosthetic heart valve of claim 10, wherein the end portion and connector are removably coupled to one another around the portion of the struts, with the portion of struts sandwiched therebetween, thereby removably coupling the commissure attachment member to the frame.

12. A prosthetic heart valve comprising:
a frame comprising:
a plurality of interconnected struts defining a plurality of circumferentially extending rows of cells arranged between an outflow end and an inflow end of the frame, wherein the plurality of circumferentially extending rows of cells comprises a first row of first cells disposed at the outflow end; and
a plurality of commissure attachment members, each commissure attachment member comprising a first end portion comprising two spaced apart leaflet attachment portions, and a second end portion secured to a portion of struts of the plurality of interconnected struts, wherein the commissure attachment member is configured with a laterally inward bias such that the two leaflet attachment portions are held adjacent one another in a first position and move laterally outward and away from one another to a second position in response to a force over a threshold applied to the two leaflet attachment portions; and
a plurality of leaflets disposed within the frame, wherein each leaflet comprises opposing commissure tabs disposed on opposite sides of the leaflet and a cusp edge portion extending between the opposing commissure tabs, and wherein at each commissure attachment member, a first commissure tab of a first of two adjacent leaflets is secured to a first leaflet attachment portion of the two leaflet attachment portions and a second commissure tab of a second of the two adjacent leaflets is secured to a second leaflet attachment portion of the two leaflet attachment portions, thereby forming a commissure of the prosthetic heart valve.

13. The prosthetic heart valve of claim 12, wherein in the first position the two leaflet attachment portions of each commissure attachment member are spaced laterally apart by a first amount, and wherein in the second position the two leaflet attachment portions are spaced laterally apart by a second amount that is greater than the first amount.

14. The prosthetic heart valve of claim 12, wherein the two spaced apart leaflet attachment portions are axially extending posts, relative to a central longitudinal axis of the frame, and wherein, for each commissure attachment member, the first commissure tab is secured around the first leaflet attachment portion and the second commissure tab is secured around the second leaflet attachment portion.

15. The prosthetic heart valve of claim 12, wherein the portion of struts to which the second end portion is secured includes one or more angled struts that at least partially define a respective first cell of the first row of first cells, and wherein the two leaflet attachment portions of each commissure attachment member are disposed within the respective first cell of the first row of first cells.

16. A commissure attachment member for a frame of a prosthetic heart valve, comprising:
a first end portion comprising two spaced apart leaflet attachment portions, wherein each leaflet attachment portion is configured to be coupled to a leaflet of the prosthetic heart valve; and
a second end portion that is configured to be secured to a portion of struts of the frame of the prosthetic heart valve, wherein the commissure attachment member is configured with a laterally inward bias such that the two leaflet attachment portions are held adjacent one another in a first position and move laterally outward and away from one another to a second position in response to a force over a threshold applied to the two leaflet attachment portions.

17. The commissure attachment member of claim 16, wherein in the first position the two leaflet attachment portions are spaced laterally apart by a first amount, and wherein in the second position the two leaflet attachment portions are spaced laterally apart by a second amount that is greater than the first amount.

18. The commissure attachment member of claim 16, wherein each commissure attachment member comprises an intermediate portion disposed between the first end portion and the second end portion, and wherein the intermediate portion comprises two opposing curved portions that curve laterally outward from the two leaflet attachment portions, in opposite directions from one another.

19. The commissure attachment member of claim 16, wherein the second end portion comprises a plurality of spaced apart protrusions, and wherein each commissure attachment member further comprises a connector comprising a plurality of spaced apart cavities, wherein each cavity of the plurality of spaced apart cavities is shaped to receive a respective protrusion of the plurality of spaced apart protrusions such that the connector mates with the second end portion.

20. The commissure attachment member of claim 19, wherein the second end portion and connector are configured to be removably coupled to one another around the portion of the struts, with the portion of struts sandwiched therebetween, thereby removably coupling the commissure attachment member to the portion of struts.
